(19) United States
(12) Patent Application Publication
(10) Pub. No.: US 2011/0028866 A1
(43) Pub. Date: Feb. 3, 2011

(54) Ultrasound Apparatus for Creating and Delivering Therapeutic Solutions

(75) Inventor: Eilaz BABAEV, Minnetonka, MN (US)

Correspondence Address: Bacoustics, LLC, 5929 BAKER ROAD, SUITE 470, MINNETONKA, MN, 55345, US

(73) Assignee: BACOUSTICS LLC, Minnetonka, MN (US)

(21) Appl. No.: 12/834,514

(22) Filed: Jul. 12, 2010

Related U.S. Application Data
(63) Continuation-in-part of application No. 11/777,934, filed on Jul. 13, 2007, now Pat. No. 7,753,285.

(51) Int. Cl. A61N 7/00

(52) U.S. Cl. 601/2

(57) ABSTRACT

The present invention relates to an ultrasound device and method for treating a wound by creating a therapeutic combination of materials with ultrasonic waves and delivering that combination into the wound bed. The apparatus comprises a horn having an internal chamber including a back wall, a front wall, and at least one side wall, a radiation surface at the horn's distal end, at least one channel opening into the chamber. Within the internal chamber of the horn the healing factor to be delivered to the wound is mixed with a suitable carrying agent to create a therapeutic solution. Affixed to the distal end of the horn is a cavitation chamber capable of deforming downwards that receives the therapeutic solution. The downward deformation of the cavitation chamber applies pressure to the therapeutic solution contained within the chamber pushing the solution into the wound bed.

### CROSS REFERENCE TO RELATED APPLICATIONS

This application is a continuation-in-part of U.S. patent application Ser. No. 11/777,934 filed Jul. 13, 2007, the teachings of which are hereby incorporated by reference.

### BACKGROUND OF THE INVENTION

1. Field of the Invention

The present invention relates to an apparatus for treating a wound by creating a therapeutic combination of materials with ultrasonic waves and delivering that combination into the wound bed.

2. Description of the Related Art

When confronted with wounded tissue, physicians and similar practitioners of medical arts have numerous devices and methods at their disposal. For instance, exposing the wound to oxygen may bring about a therapeutic effect. Methods of delivering oxygen to wounds have been developed and are implemented by various devices and compounds. The methods include placing the wound within an oxygen rich environment as to facilitate the diffusion of oxygen from the environment into the wound. Oxygen releasing compounds have also been placed over wounds as to allow for the diffusion of oxygen from the compound into the wound.

Administering pharmaceuticals to the wound may also be utilized to treat wounded tissue by providing various therapeutic benefits. For instance, a therapeutic benefit may be obtained by utilizing pharmaceuticals to prevent an infection from developing in the wounded tissue. Specifically, keeping the wound in an infection free state can be accomplished by administering various anti-microbial agents such as, but not limited to, antiseptics, antibiotics, antiviral agents, antifungal agents, or any combination thereof. Administering various growth factors to the wounded tissue may also elicit a therapeutic benefit by promoting the growth of new tissue.

In extreme situations, the practitioner may have to resort to surgery to treat the wounded tissue. Grafting transplanted and/or bioengineered tissue onto the wounded may be necessary with severe wounds.

More experimental treatments, such as exposing the wounded tissue to ultraviolet light, electricity, and/or ultrasound, are also available to the practitioner. For example, U.S. Pat. Nos. 6,478,754, 6,761,729, 6,533,803, 6,569,099, 6,663,554, and 6,960,173 teach methods and devices utilizing an ultrasound generated spray to treat wounded tissues. Methods and devices utilizing indirect contact with the wounded tissue via a liquid aerosol are disclosed in U.S. Pat. Nos. 7,025,735 and 6,916,296.

### SUMMARY OF THE INVENTION

Treating severe and chronic wounds in at risk populations such as the elderly, diabetics, and individuals with compromised immune systems can be especially difficult. The presence of an unhealed wound is an unnecessary burden. The pain produced by such wounds may disable the patient, thereby reducing their quality of life. An unhealed wound's susceptibility to infection increases the patient's morbidity and mortality. The increased prevalence of drug resistant infectious agents often seen within institutions, such as hospitals and care homes, where at risk patients inhabit or often frequent further increases patient morbidity and mortality.

Proper wound healing requires the tissue comprising the wound bed receives nutrients and other healing promoting factors. Generally, such factors are delivered to the wound bed through the circulatory system. The blood supply to wounded tissue, unfortunately, is often diminished or compromised. Consequently, the amount of the healing promoting factors reaching wounded tissue is often reduced.

The present invention provides an ultrasound apparatus capable of delivering various healing factors to a wound bed. The apparatus comprises a horn having an internal chamber including a back wall, a front wall, and at least one side wall, a radiation surface at the horn's distal end, at least one channel opening into the chamber, and a channel originating in the front wall of the internal chamber and terminating in the radiation surface. Within the internal chamber of the horn the healing factor to be delivered to the wound is mixed with a suitable carrying agent to create a therapeutic solution. Affixed to the distal end of the horn is a cavitation chamber capable of deforming downwards that receives the therapeutic solution. The downward deformation of the cavitation chamber applies pressure to the therapeutic solution contained within the chamber pushing the solution into the wound bed.

The therapeutic solution is created within the internal chamber of the ultrasound horn by the action of ultrasonic vibrations mixing the healing factor with the carrying agent. Connected to the horn's proximal end, a transducer powered by a generator induces ultrasonic vibrations within the horn. Traveling down the horn from the transducer to the horn's radiation surface, the ultrasonic vibrations are released into the chamber from the chamber's back wall. As the vibrations travel through the chamber the healing factor and carrying agent within the chamber are agitated and thereby mixed together. Upon reaching the front wall of the chamber, the ultrasonic vibrations are reflected back into the chamber, like an echo. The ultrasonic vibrations echoing off the front wall pass through the chamber a second time, further mixing the healing factor and carrying agent.

As the vibrations travel back-and-forth within the chamber, they may strike protrusions located on the side walls of the chamber. After striking a protrusion, the vibrations are scattered about the chamber. Consequently, some of the vibrations echoing off the side wall protrusions may be reflected back towards the wall of the chamber from which they originated. Some the vibrations may continue on towards the opposite the wall of the chamber. The remainder of the vibrations may travel towards another side wall of the chamber where they will be scattered once more by the protrusion. Therefore, the echoing action of ultrasonic vibrations within the chamber is enhanced by the protrusions on the side walls of the chamber. Emitting ultrasonic vibrations into the chamber from their distal facing edges, the protrusions within the inner chamber may also enhance the mixing of the healing factor and carrying agent by increasing the amount of ultrasonic vibrations within the chamber.

The protrusions may be formed in a variety of shapes such as, but not limited to, convex, spherical, triangular, rectangular, polygonal, and/or any combination thereof. The protrusions may be discrete elements. Alternatively, the protrusions may be discrete bands encircling the internal chamber. The protrusions may also spiral down the chamber similar to the threading within a nut.

The healing factor and carrying agent mixed within the chamber by the echoing and scattered ultrasonic vibrations may be any fluid and additional substance the attending physician believes will promote healing of the wound to be treated. For instance, oxygen may be utilized as the healing factor and saline as the carrying agent. Oxygen is essential for many important aspects of the healing process. For example, oxygen is required for cellular respiration, the process by which cells produce the energy needed to heal the wound. Normally oxygen delivered to tissues is transported through the blood attached to hemoglobin. As the blood travels past the cells of the body, the oxygen disassociates from hemoglobin and enters the blood plasma, a fluid similar to saline. The oxygen then diffuses from the plasma into the cells of the body. Oxygen dissociation from hemoglobin and diffusion into cells is a result of the entropic drive to achieve an equal concentration of oxygen in the blood and cells, or equilibrium. Thus, oxygen absorption by cells is driven by the difference in oxygen partial pressure between the blood and cells.

By passing oxygen and saline through the internal chamber of the horn a therapeutic solution similar to oxygen containing blood plasma can be created. When delivered deep into the wound bed by the downward deformation of the cavitation chamber receiving the solution from the horn, the oxygen within the solution may diffuse into the cells of the wound bed by the entropic drive to achieve equilibrium as it would from blood plasma.

The cavitation chamber attached to the distal end horn may be made capable of downward deformation by forming the chamber from a subtle material. It is also possible to provide for downward deformation by including collapsible sides on the cavitation chamber. For example, the cavitation chamber could be provided with inter sliding segments permitting telescoping of the chamber or the sides of the chamber could include pleats similar to the prototypical accordion. It also possible that the sides of the chamber may be fixed and downward deformation accomplished by the movement of a depressible member.

Relieving the downward deformation of the chamber may withdraw the solution from the wound bed. As the solution exits the wound bed any waste or debris that may have entered the solution will be carried away from the cells. By delivering nutrients and extracting waste from the wound bed as it exits and enters the wound bed due to the downward and upward deformation of the cavitation chamber, the therapeutic solution may supplement the diminished or comprised blood supply to the wound bed.

The ability of the therapeutic to enter the wound bed may be increased by cavitations induced within the cavitation chamber by ultrasonic energy emitted from the horn's radiation surface. As a wound persists a diverse amount of material may build up over the wound inhibiting the ability of the therapeutic solution to penetrate into the wound bed. For instance, foreign substances such as, but not limited to, dirt, debris, and/or infectious agents may collect within the wound. In the case of an infectious agent such as, but not limited to, a bacteria, a bacterial laden biofilm may develop over the wound. As the infection increases in severity, the wound may become covered with gangrenous tissue.

Additionally the compromised blood supply may result in ischemic tissue forming over the wound that inhibits entry of the therapeutic solution to the wound bed. Ischemia may also be the result of various conditions such as, but not limited, diabetes and/or various vascular diseases. As the ischemia persists, the tissue becomes deprived of vital nutrients required for growth and/or survival, and thus may eventually become devitalized. Failing to receive required nutrients, the devitalized tissue may eventually slip into a non-viable state. The non-viable tissue may begin a process of necrosis and/or apoptosis in which the cells of the non-viable tissue release various factors the digest and/or degrade the tissue. Destroying itself, the non-viable tissue becomes necrotic tissue. If the degradation and/or digestive process continues beyond the point of cellular death, the necrotic tissue may become slough. However, it is also possible that digestion and/or degradation stops with cellular death as to create an eschar over the wound. Regardless of how far the tissue progresses from ischemia and/or devitalization to slough and/or eschar, the dead and dying tissue may block access to the wound bed.

Material blocking entry of the therapeutic solution may also be generated by the wound itself. For instance, in response to an inflammation brought about by the presence of foreign substances and/or trauma an exudate may be secreted. As the secretion of exudates persists, the wound may become covered by various proteins and/or other molecules manufactured by the body. Secretion of a fibrinous exudate, for example, may lead to a build up fibrin over the wound. Regardless of the type of exudate secreted and/or built up over the wound, this body generated material may block access to the wound bed.

Ultrasound energy released from the radiation surface of the horn into the therapeutic solution or other liquid held within the cavitation chamber results in the formation of tiny bubbles, i.e. cavitations. Conceptually, this phenomenon is similar to inducing water to boil by applying heat. However, the induction of cavitations by ultrasound energy is not dependant upon heating the solution or other liquid to its boiling point. As such, the induction of cavitations is not dependent upon the transfer of thermal energy.

After spontaneously forming within the liquid held within the cavitation chamber the cavitations randomly explode and/or collapse. An exploding and/or collapsing cavitation releases energy into the liquid surrounding it. Furthermore, the explosion and/or collapse of a cavitation induces a pressure change within the volume of the liquid surrounding the cavitation. The pressure change and/or energy released may disrupt build up over the wound inhibiting the ability of the therapeutic solution to penetrate into the wound bed. Thus, the cavitations induced within the cavitation chamber over the wound by ultrasound energy emitted from the radiation surface may increase the ability of the therapeutic solution to enter the wound bed.

The cavitations induced over the wound may also increase the ability of the therapeutic solution to enter the wound bed by disrupting the association betweens cells as to widen the gaps between cells. Additionally, the ultrasonic energy released from the radiation surface may perturb cellular membranes as to increase the movement of healing factors from the solution into the cells.

It should be noted and appreciated that other benefits, mechanisms of action, and/or mechanisms of operation, in addition to those listed, may be elicited by with the present invention. The mechanisms of action and mechanisms of operation presented herein are strictly theoretical and are not meant in any way to limit the scope this disclosure and/or the accompanying claims.

### BRIEF DESCRIPTION OF THE DRAWINGS

FIG. 1 is a cross section of one embodiment of an ultrasound apparatus according to the present invention.

FIG. 2 illustrates a cross-sectional view of an alternative embodiment of an ultrasound apparatus according to the present invention with ultrasonic lens within back wall and an ultrasonic lens within front wall of the internal chamber of the horn containing concave portions.

FIG. 3 illustrates varying radiation surfaces that may be used with the present invention.

### DETAILED DESCRIPTION OF THE INVENTION

Preferred embodiments of the ultrasound apparatus comprising the present invention are illustrated throughout the figures and described in detail below.

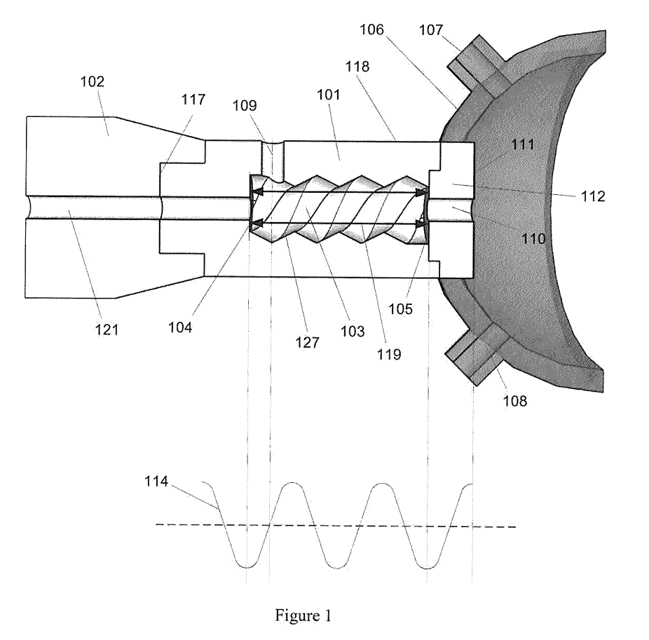

FIG. 1 illustrates an embodiment of the ultrasound apparatus comprising a horn 101 and an ultrasound transducer 102 attached to the proximal end 117 of horn 101 powered by generator (not shown). As ultrasound transducers and generators are well known in the art they need not and will not, for the sake of brevity, be described in detail herein. Ultrasound horn 101 comprises a proximal end 117, a radiation surface 111 opposite proximal end 117, and at least one radial surface 118 extending between proximal surface 117 and radiation surface 111. Within horn 101 is an internal chamber 103 containing a back wall 104, a front wall 105, at least one side wall extending between back wall 104 and front wall 105, and protrusion 127 located on the side wall and extending into chamber 103. Horn 101 and chamber 103 may be cylindrical, as depicted in FIG. 1. Horn 101 and chamber 103 may also be constructed in other shapes and the shape of chamber 103 need not correspond to the shape of horn 101.

As to induce vibrations within horn 101, ultrasound transducer 102 may be mechanically coupled to proximal end 117. Mechanically coupling horn 101 to transducer 102 may be achieved by mechanically attaching (for example, securing with a threaded connection), adhesively attaching, and/or welding horn 101 to transducer 102. Other means of mechanically coupling horn 101 and transducer 102, readily recognizable to persons of ordinary skill in the art, may be used in combination with or in the alternative to the previously enumerated means. Alternatively, horn 101 and transducer 102 may be a single piece. When transducer 102 is mechanically coupled to horn 101, driving transducer 102 with an electrical signal supplied from the generator induces ultrasonic vibrations 114 within horn 101. If transducer 102 is a piezoelectric transducer, then the amplitude of the ultrasonic vibrations 114 traveling down the length of horn 101 may be increased by increasing the voltage of the electrical signal driving transducer 102.

As the ultrasonic vibrations 114 travel down the length of horn 101, back wall 104 oscillates back-and-forth. The back-and-forth movement of back wall 104 induces the release of ultrasonic vibrations into chamber 103. Positioning back wall 104 such that at least one point on back wall 104 lies approximately on an antinode (point of maximum deflection) of the ultrasonic vibrations 114 passing through horn 101 may maximize the amount and/or amplitude of the ultrasonic vibrations emitted into chamber 103. Preferably, the center of back wall 104 lies approximately on an antinode of the ultrasonic vibrations 114. The ultrasonic vibrations emanating from back wall 104, represented by arrows 119, travel towards the front of chamber 103. When the ultrasonic vibrations 119 strike front wall 105 they echo off it, and thus are reflected back into chamber 103. The reflected ultrasonic vibrations 119 then travel towards back wall 104. Traveling towards front wall 105 and then echoing back towards back wall 104, ultrasonic vibrations 119 travel back and forth through chamber 103 in an echoing pattern. As to maximize the echoing of vibrations 119 off front wall 105, it may be desirable to position front wall 105 such that at least one point on it lies on an antinode of the ultrasonic vibrations 114. Preferably, the center of front wall 105 lies approximately on an antinode of the ultrasonic vibrations 114.

The incorporation of protrusions 127 enhances ultrasonic echoing within chamber 103 by increasing the amount of ultrasonic vibrations emitted into chamber 103 and/or by providing a larger surface area from which ultrasonic vibrations echo. The distal or front facing edges of protrusions 127 may emit ultrasonic waves into chamber 103 when the ultrasound transducer 102 is activated. The proximal, or rear facing, and front facing edges of protrusions 127 reflect ultrasonic waves striking the protrusions 127. Emitting and/or reflecting ultrasonic vibrations into chamber 103, protrusions 127 increase the complexity of the echoing pattern of the ultrasonic vibrations within chamber 103. The specific protrusions 127 depicted in FIG. 1 comprise a triangular shape and spiral down the chamber similar to the threading within a nut. The protrusions may be formed in a variety of shapes such as, but not limited to, convex, spherical, triangular, rectangular, polygonal, and/or any combination thereof. In the alternative or in combination to spiraling down the chamber, the protrusions may be discrete bands encircling the chamber. In combination or in the alternative, the protrusions may also be discrete elements secured to a side wall of chamber that do not encircle the chamber. In the alternative or in combination, the protrusions may be integral with side wall or walls of the chamber.

The healing factor or carrying agent to be mixed to create the therapeutic solution enter chamber 103 of the embodiment depicted in FIG. 1 through channel 109 originating in radial surface 118 and opening into chamber 103. Preferably, channel 109 encompasses a node of the ultrasonic vibrations 114 traveling down the length of the horn 101 and/or emanating from back wall 104. In the alternative or in combination, channel 109 may originate in radial surface 118 and open at back wall 104 into chamber 103. Upon exiting channel 109, the material flows through chamber 103 and becomes mixed with the other material entering chamber 103 through channel 121 originating in proximal end 117 and opening within back wall 104 to create a therapeutic solution. The therapeutic solution created then exits chamber 103 through channel 110, originating within front wall 105 and terminating within radiation surface 111. It is preferable if at least one point on radiation surface 111 lies approximately on an antinode of the ultrasonic vibrations 114 passing through horn 101.

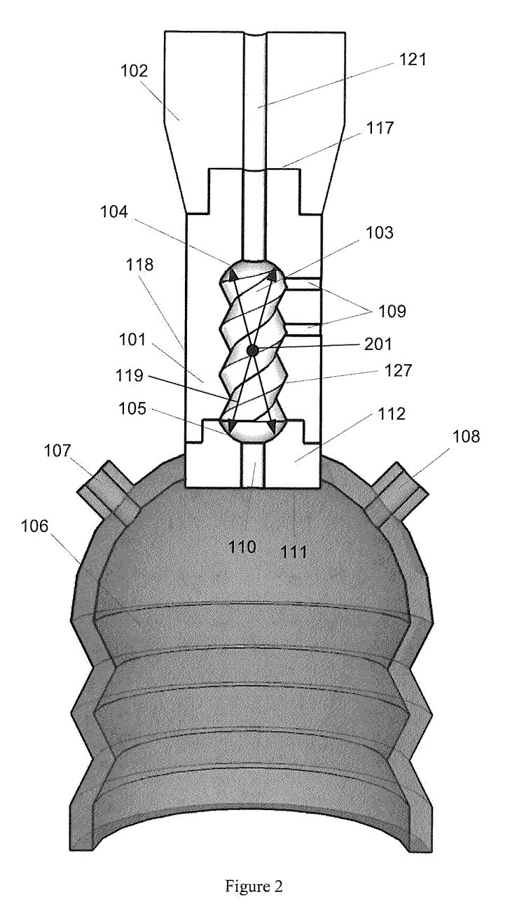

Alternative embodiments of an ultrasound horn 101 in accordance with the present invention may possess a single channel 109 opening within side wall 113 of chamber 103. If multiple channels 109 are utilized, they may be aligned along the central axis 120 of horn 101, as depicted in FIG. 2. Channels 109 may also be located on different platans, as depicted in FIG. 2, and/or the same platan.

A single channel may be used to deliver the carrying agent and healing factor to be mixed to create the therapeutic solution into chamber 103.

The therapeutic solution exiting horn 101 is received by a cavitation chamber 106 capable of downward deformation affixed to the distal end of horn 101. The cavitation chamber 106 comprises an inner cavity open at its base capable of holding a therapeutic solution and/or other liquid against the wound. Cavitation chamber 106 may, but need not, envelope radiation surface 111. Thus, radiation surface 111 may be located within cavitation chamber 106, as depicted in FIG. 1, or outside cavitation chamber 106. If located outside of cavitation chamber 106 it is advisable that a material transparent to ultrasonic energy be position be the radiation surface and the inner cavity of cavitation chamber 106.

Cavitation chamber 106 may be integral with the horn 101. Alternatively, cavitation chamber 106 may be a separate piece mechanically affixed (for example, secured with a threaded connector), adhesively affixed, and/or welded to the distal end of horn 101. Other means of attaching cavitation chamber 106 to horn 101, readily recognizable to persons of ordinary skill in the art, may be used in combination with or in the alternative to the previously enumerated means. The means of attaching cavitation chamber 106 to horn 101 may be such as to allow cavitation chamber 106 to be removed and replaced. A removable cavitation chamber enables the size and/or configuration of the chamber to be adjusted as to conform to the wound being treated.

Cavitation chamber 106 may be constructed entirely from an autoclavable metallic and/or plastic substance as to permit sterilization after use. Cavitation chamber 106 may also be constructed entirely or in part from a supple material, such as, but not limited to, a polymer or plastic as to enable downward deformation of the chamber. The material used to construct the supple portions of cavitation chamber 106 should be sufficiently inelastic such that the downward deformation of cavitation chamber 106 applies pressure to therapeutic solution contained within in its inner cavity as to push the solution into the wound bed.

Constructing cavitation chamber 106 in whole or in part of a supple material allows cavitation chamber 106 to conform to the contours of the patient's body as to form a better seal to retain the solution during treatment. Adding a liquid sealant to the base of cavitation chamber 106 further enhances the seal between base of the chamber and the patient's skin. The liquid sealant may comprise, but is not limited to, silicon gel, medical gel, medical adhesive, or water. Furthermore, constructing cavitation chamber 106 in whole or in part of a supple material allows an alternating general positive and negative pressure to be applied to the fluids within the cavity of the chamber by pushing down and lifting up on the device; similar in motion and effect to a plumber using a plunger to repair a clogged toilet. The alternating pressure pushes and pulls the therapeutic solution into and out of the wound bed enabling the delivery of the healing factor and removal of waste.

As to facilitate plunging, the base of cavitation chamber 106 may contain pleats similar to the prototypical accordion, as depicted in FIG. 2.

As seen in FIG. 1, cavitation chamber 106 may contain a feed port 107 and extraction port 108 permitting fluids to be fed into and extracted from its inner cavity. If the induction of cavitations over the surface of the wound is desired, then it is advisable to completely submerge the point at which ultrasonic energy enters the cavity of chamber 106 with a fluid within the cavity. This can be accomplished by filling the chamber with therapeutic solution received from the horn while any air present in the chamber is allowed to escape via extraction port 108. Alternatively, the chamber may be filled with a liquid via feed port 107 to force air with the chamber out via extraction port 108.

As to simplify manufacturing, ultrasound horn 101 may further comprise cap 112 attached to its distal end. Cap 112 may be mechanically attached (for example, secured with a threaded connector), adhesively attached, and/or welded to the distal end of horn 101. Other means of attaching cap 112 to horn 101, readily recognizable to persons of ordinary skill in the art, may be used in combination with or in the alternative to the previously enumerated means. Attaching cap 112 to the present invention at approximately a nodal point of the ultrasonic vibrations 114 passing through horn 101 may help prevent the separation of cap 112 from horn 101 during operation. Comprising front wall 105, channel 110, and radiation surface 111, a removable cap 112 permits the dispersion of ultrasonic energy into the fluid within cavitation chamber 106 to be adjusted depending on need and/or circumstances.

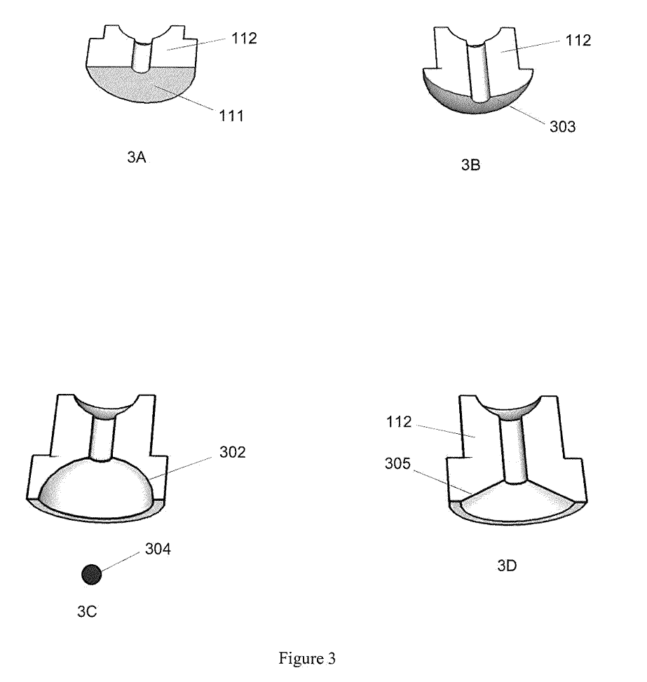

FIG. 3 illustrates alternative embodiments of cap 112 containing varying radiation surfaces. FIG. 3A depicts radiation surfaces 111 comprising a planar face producing a roughly column-like transmission of ultrasonic energy into the cavitation chamber. Horns used with the present invention possessing a planar radiation surface may be tapered such that it the radiation surface is narrower than the width of the horn in at least one dimension oriented orthogonal to the central axis of the horn. The majority of cavitations induce by ultrasonic energy emanating from the radiation surfaces 111 depicted in FIG. 3A is confined to the surface of the wound directly below the radiation surface.

The ultrasonic energy emitted from the convex portion 303 of the radiation surface 111 depicted in FIG. 3B is directed radially and longitudinally away from radiation surface 111. The resulting dispersion of ultrasonic energy throughout the liquid held within cavitation chamber 106 may lead to induction of cavitations over the entire surface of the wound encased by chamber 106. Conversely, the ultrasonic energy emanating from the concave portion 302 of the radiation surface 111 depicted in FIG. 3C is focused towards point 304. This will result in the majority of cavitations induced on the surface of the wound occurring within a confined area on the wound surface. Maximizing the focusing of the emitted ultrasonic energy towards point 304 may be accomplished by constructing radiation surface 111 such that point 304 is the focus of an overall parabolic configuration formed in at least two dimensions by concave portion 302. The radiation surface 111 may also possess a conical portion 305 as depicted in FIG. 3D. Ultrasonic energy emanating from the conical portion 305 is directed inwards leading to a gradient induction of cavitations over the surface of the wound. The radiation surface may possess any combination of the above mentioned configurations such as, but not limited to, an outer concave portion encircling an inner convex portion and/or an outer planar portion encompassing an inner conical portion.

Regardless of the configuration of the radiation surface, adjusting the amplitude of the ultrasonic vibrations traveling down the length of the horn may be useful in achieving the desired pattern and amount cavitations over the surface of the wound.

FIG. 2 illustrates a cross-sectional view of an alternative embodiment of the ultrasound horn that may be used in the present invention further comprising an ultrasonic lens within back wall 104 and an ultrasonic lens within front wall 105 containing concave portions. If the concave portion of the lens within back wall 104 form an overall parabolic configuration in at least two dimensions, then the ultrasonic vibrations depicted by arrows 119 emanating from the lens will travel in a pattern of convergence towards the parabola's focus 201. As the ultrasonic vibrations 119 converge at focus 201, the ultrasonic energy carried by vibrations 119 may become focused at focus 201. After converging at focus 201, the ultrasonic vibrations 119 diverge and continue towards front wall 105. After striking the concave portion of the lens within front wall 105, ultrasonic vibrations 119 are reflected back into chamber 103. If concave portions of the lens within front wall 105 form an overall parabolic configuration in at least two dimensions, the ultrasonic vibrations 119 echoing backing into chamber 103 may travel in a pattern of convergence towards the parabola's focus. Converging as they travel towards front wall 105 and then again as they echo back towards back wall 104, ultrasonic vibrations 119 travel back and forth through chamber 103 in a converging echoing pattern.

In addition to focusing the ultrasonic vibrations 119 and/or the ultrasonic energy they carry, ultrasonic lens with the front and back wall of the internal chamber 103 direct the ultrasonic vibrations 119 towards the side walls of the chamber. As such, an increased amount of ultrasonic vibrations emanating from back wall 104 and/or reflecting off front wall 105 strike side wall 113 and become scattered by protrusions 127.

In the embodiment illustrated in FIG. 2 the parabolas formed by concave portions of the lenses within the front wall 105 and back wall 104 have a common focus 201. In the alternative, the parabolas may have different foci. However, by sharing a common focus 201, the ultrasonic vibrations 119 emanating and/or echoing off the parabolas and/or the energy the vibrations carry may become focused at focus 201. The materials passing through chamber 103 are therefore exposed to the greatest concentration of the ultrasonic agitation at focus 201. Consequently, the ultrasonically induced mixing of the fluids is greatest at focus 201. Positioning focus 201, or any other focus of a parabola formed by the concave portions of lenses within the front wall 105 and back wall 104, at point downstream of the entry of at least two materials into chamber 103 may maximize the mixing of the materials.

Although not illustrated an alternative embodiment of the ultrasound atomizing and/or mixing apparatus may have a lens within back wall 104 and/or a lens within front wall 105 containing convex portions. Ultrasonic vibrations emanating from convex portions of a back wall lens will travel in a dispersed reflecting pattern towards front wall 105 in the following manner: The ultrasonic vibrations will be first directed towards side wall 113 at varying angles of trajectory. The ultrasonic vibrations will then reflect off side wall 113 and become scattered by protrusions 127. The scattered ultrasonic vibrations may then travel back towards back wall 104, continue on towards front wall 105, and/or become scattered again by protrusions 127 on another region of side wall 113. Likewise, when the ultrasonic vibrations strike convex portions with a lens on front wall 105, they will echo back into chamber 103 towards side wall 113 and become scattered. As such, some of the ultrasonic vibrations echoing off convex portions within a front lens may continue on towards back wall 104 after striking side wall 113. Some of the echoing ultrasonic vibrations may travel back towards front wall 105. The remainder may strike another region of side wall 113 and become scattered again.

It should be appreciated that the configuration of the chamber's front wall lens need not match the configuration of the chamber's back wall lens. Furthermore, the lenses within the front and/or back walls of the chamber may comprise any combination of the above mentioned configurations such as, but not limited to, an outer concave portion encircling an inner convex portion.

The horn may be capable of vibrating in resonance at a frequency of approximately 16 kHz or greater. The ultrasonic vibrations traveling down the horn may have an amplitude of approximately 1 micron or greater. It is preferred that the horn be capable of vibrating in resonance at a frequency between approximately 20 kHz and approximately 200 kHz. It is recommended that the horn be capable of vibrating in resonance at a frequency of approximately 30 kHz.

The signal driving the ultrasound transducer may be a sinusoidal wave, square wave, triangular wave, trapezoidal wave, or any combination thereof.

It should be appreciated that elements described with singular articles such as “a”, “an”, and/or “the” and/or otherwise described singularly may be used in plurality. It should also be appreciated that elements described in plurality may be used singularly.

Although specific embodiments of apparatuses and methods have been illustrated and described herein, it will be appreciated by those of ordinary skill in the art that any arrangement, combination, and/or sequence that is calculated to achieve the same purpose may be substituted for the specific embodiments shown. It is to be understood that the above description is intended to be illustrative and not restrictive. Combinations of the above embodiments and other embodiments as well as combinations and sequences of the above methods and other methods of use will be apparent to individuals possessing skill in the art upon review of the present disclosure.

The scope of the claimed apparatus and methods should be determined with reference to the appended claims, along with the full scope of equivalents to which such claims are entitled.

I claim:

1. An apparatus characterized by:
a. a proximal end;
b. a radiation surface opposite the proximal end;
c. at least one radial surface extending between the proximal end and the radiation surface;
d. an internal chamber containing:
i. a back wall;
ii. a front wall; and
iii. at least one side wall extending between the back wall and the front wall;
e. at least one channel originating in a surface other than the radiation surface and opening into the internal chamber;
f. a channel originating in the front wall of the internal chamber and terminating in the radiation surface;
g. at least one protrusion on a side wall of the chamber and extending into the chamber; and
h. a chamber attached to the distal end capable of receiving fluids from the chamber and downward deformation.

2. The apparatus according to claim 1 further characterized by at least one point on the back wall of the chamber lying approximately on an anti-node of the vibrations of the apparatus.

3. The apparatus according to claim 1 further characterized by at least one point on the radiation surface lying approximately on an anti-node of the vibrations of the apparatus.

4. The apparatus according to claim 1 further characterized by at least one point on the front wall of the chamber lying approximately on a anti-node of the vibrations of the apparatus.

5. The apparatus according to claim 1 further characterized by the channel opening into the chamber originating in a radial surface and opening into a side wall of the internal chamber approximately on a node of the vibrations.

6. The apparatus according to claim 1 further characterized by a transducer attached to the proximal surface.

7. The apparatus according to claim 6 further characterized by a generator to drive the transducer.

8. The apparatus according to claim 1 characterized by the channel opening into the chamber originating in the proximal end and opening into the back wall of the internal chamber.

9. The apparatus according to claim 1 further comprising an ultrasonic lens within the back wall of the chamber.

10. The apparatus according to claim 9 further comprising one or a plurality of concave portions within the lens within the back wall that form an overall parabolic configuration in at least two dimensions.

11. The apparatus according to claim 1 further comprising an ultrasonic lens within the front wall of the chamber.

12. The apparatus according to claim 11 further comprising one or a plurality of concave portions within the lens within the front wall that form an overall parabolic configuration in at least two dimensions.

13. The apparatus according to claim 1 further comprising at least one planar portion within the radiation surface.

14. The apparatus according to claim 1 further comprising a central axis extending from the proximal surface to the radiation surface and a region of the radiation surface narrower than the width of the apparatus in at least one dimension oriented orthogonal to the central axis.

15. The apparatus according to claim 1 further comprising at least one concave portion within the radiation surface.

16. The apparatus according to claim 1 further comprising at least one convex portion within the radiation surface.

17. The apparatus according to claim 1 further comprising at least one conical portion within the radiation surface.

18. The apparatus according to claim 1 wherein the radiation surface is within the chamber.

19. The apparatus according to claim 1 further comprising a port within the chamber.
